(19) United States
(12) Patent Application Publication
(10) Pub. No.: US 2026/0287574 A1
(43) Pub. Date: Sep. 24, 2026

(54) APPLICATION OF A BRAIN ORGANOID MODEL FOR SIMULATING THE DAMAGE OF ENVIRONMENTAL POLLUTANTS TO BRAIN DEVELOPMENT DURING EMBRYONIC PERIOD

(71) Applicant: Tianjin University, Tianjin (CN)

(72) Inventors: Liqun CHEN, Tianjin (CN); Yue CHEN, Tianjin (CN); Shiqun CHEN, Tianjin (CN)

(73) Assignee: Tianjin University, Tianjin (CN)

(21) Appl. No.: 18/712,712

(22) PCT Filed: Feb. 19, 2024
(86) PCT No.: PCT/CN2024/077602

§ 371 Date: Apr. 21, 2026

(30) Foreign Application Priority Data
Oct. 28, 2023 (CN) 202311411541.9

(51) Int. Cl. G01N 33/50; C12N 5/00; C12N 5/0793

(52) U.S. Cl. CPC G01N 33/5014; C12N 5/0031; C12N 5/0619; G01N 33/5058; G01N 33/5088; C12N 2501/727; C12N 2506/02; C12N 2513/00

(57) ABSTRACT

The present invention discloses the application of a brain organoid model for simulating the study of embryonic environmental pollutants on brain development damage, which belongs to the field of environmental exposure prevention and treatment technology. The brain organoid model includes at least group cells (SOX2⁺ cells), neurons (MAP2⁺ cells), deep neurons (TBR1⁺ cells) and immature neurons (TUJ1⁺ cells). The simulated exposure of environmental pollutants in brain organoids constructed by the invention and the subsequent mechanism exploration and treatment intervention research are a promising method to study the effects of environmental pollutants on human health. Compared with traditional toxicological testing methods, this technology has a number of advantages, helping researchers better understand the impact of environmental-related diseases on human health, and helping to develop new strategies for the prevention and treatment of these diseases.

### TECHNICAL FIELD

The present invention belongs to the technical field of environmental exposure prevention and treatment, specifically, it involves an application of a brain organoid model for simulating the damage of environmental pollutants to brain development during embryonic period.

### BACKGROUND ART

The study of the effects of environmental exposure on the humans depends largely on technology, from monitoring equipment that measures air and water quality to complex imaging techniques that enable researchers to visualize the effects of environmental toxins on tissues and organs. Despite advances in technology, there are still several problems in the study of the effects of environmental exposure on humans. It includes: limitations of biomonitoring: although biomonitoring can provide valuable information about exposure levels, it does not always provide a clear link between exposure and health outcomes. This is because different people may have different responses to the same level of exposure, depending on factors such as age, genetics, and overall health. Limited availability of environmental sensors: while environmental sensors can provide real-time data on pollutant levels, they are not widely available or affordable for researchers. This may limit their use in heavily polluted areas. Ethical aspects considerations: some techniques used to study the effects of environmental exposure on the humans, such as exposing the humans to environmental toxins, can cause ethical problems. It is important to ensure that research is conducted in an ethical and responsible manner. Lack of longitudinal studies: many studies on the effects of environmental exposure on the human body are cross-sectional, meaning that they provide only feedback on exposure and health outcomes at a single point in time. With the passage of time, longitudinal studies that track individuals can provide more information about the long-term effects of environmental exposure. It is difficult to attribute health outcomes to environmental exposure: attributing specific health outcomes to environmental exposure can be challenging because many factors can affect health. This may make it difficult to establish causal relationships and develop effective interventions to reduce exposure and prevent adverse health effects. In general, although technology has promoted research on the effects of environmental exposure on humans, there are still some challenges and limitations that need to be addressed. Further research is needed to develop new technologies and methods to better understand the complex relationship between environmental exposure and human health.

### SUMMARY OF THE INVENTION

In order to solve the problems existing in the existing technology, the present invention provides an application of a brain organoid model for simulating the damage of environmental pollutants in embryonic period on brain development, the disease model of the present invention is more accurate and predictive, with lower cost and less demand for animal experiments; it can be grown in large quantities and screened using high-throughput methods, allowing researchers to quickly and effectively test the effects of multiple environmental toxins on organoid behavior.

In order to achieve the above purpose, the technical scheme of the present invention includes:
first, the present invention provides an application of a brain organoid model for simulating the study of brain development damage caused by environmental pollutants during embryonic period.

Further, in the above technical scheme, the brain organoid model has a cerebral cortex structure, and the brain organoid model includes at least: group cells: SOX2⁺ cells, neurons: MAP2⁺ cells, deep neurons: TBR1⁺ cells and immature neurons: TUJ1⁺ cells.

Further, in the above technical scheme, an embryonic period is the human embryonic period.

Further, in the above technical scheme, the environmental pollutants include the hazards caused by production, construction or activities.

Further, in the above technical scheme, the environmental pollutants include: endocrine disrupting chemicals (EDCs), pharmaceuticals and personal care products (PPCPs), perfluorinated compounds (PFCs), brominated flame retardants (BRPs), disinfection by-products in drinking water (DBPs).

Further in the above technical scheme, a construction method of the brain organoid model includes: culturing human embryonic stem cells in serum-free human embryonic stem cell culture medium to form embryoid bodies; then adding a ROCK inhibitor, after a certain time of differentiation, the embryoids are differentiated into cortical organoids, then replaced with vitamin A-free neural differentiation medium for culture, and obtaining a product.

Further, in the above technical scheme, the human embryonic stem cells are human embryonic stem cell line H9.

Further, in the above technical scheme, the ROCK inhibitor is Y27632.

Further, in the above technical scheme, a concentration of the ROCK inhibitor is 50-60 μM.

Further, in the above technical scheme, the differentiation time is 10-12 days.

The present invention mainly relates to the exposure method of environmental pollutants and the selection of damage index, including:
a) making the pollutant contact with the brain organoids model (according to the pollutant content in gas and water to predict the exposure concentration, concentration gradient can be set up to study the concentration threshold of pollutants causing brain development damage);
b) characterizing the growth and apoptosis marker of brain organoids to determine whether environmental exposure has an effect on the growth and development of various types of cells in brain development;
c) morphological characterization of whether the whole brain organoid is damaged, and Preliminary estimating and determining whether environmental pollutants have an impact on brain structure.
d) detecting whether the important groups of cells in the growth process of brain organoids are damaged by the markers of nerve and glial cells to locate specific cell groups, so as to determine whether environmental pollutants have potential effects on brain development;
e) high-throughput sequencing to explore the mechanism of injury, such as the identification of differentially expressed genes by using transcriptome sequencing technology, and the enrichment of these genes in specific pathways, provides a starting point for understanding the potential mechanism of injury. Further examine the potential sites that these key genes may find, and further develop agonists and inhibitors that apply corresponding potential targets to reverse the pathological regulation of environmental pollutants.

### BENEFICIAL EFFECTS

The present invention finds that the brain organoid model constructed by the invention is more suitable for studying the effect of environmental pollutants on embryonic brain development than animal models. The brain organoid model is very similar to the cell clusters in the development of the cerebral cortex, which is thought to play a key role in the evolution of the size and complexity of the human cortex. Different from the rodent model, the human embryonic cerebral cortex contains a large number of specialized outer radial glial cells (oRGC) in the outer subventricular zone (oSVZ). In brain organoids, this key feature of human brain development can be replicated. Brain organoids reproduce the developmental process of neuronal marker expression in six cortical layers, which shows a developmental trajectory similar to that of human cortex. Compared with traditional research methods, the model of this disease helps researchers better understand the impact of environmental toxins on human health, and the results of this study can be used to develop corresponding diagnosis and treatment methods.

The present invention uses the brain organoids model to simulate the new paradigm of human embryonic exposure to environmental exposure, which has many advantages. The latest technologies for the study of brain organoid exposure include the maturation of brain organoids, the selection of exposures for simulated exposure (high-throughput), the use of advanced imaging techniques for characterization, and high-throughput sequencing screening methods. This new paradigm of brain organoids simulating environmental exposure is more accurate and more predictive; its cost is lower, the demand for animal testing is less; it can grow in large quantities and use high-throughput methods for screening, so that researchers can quickly and effectively test the effects of various environmental toxins on organoid behavior. In conclusion, the brain organoid-based exposure model is the most advanced method to study the effects of environmental toxins on human tissues and organs. Compared with traditional methods, it has many advantages, including higher accuracy and predictability, lower cost, high-throughput screening and personalized medical methods.

### 1. Research Effect Aspect

1) More accurate and predictive: the brain organoid model is a three-dimensional structure, which is very suitable for imitating the structure and function of the human brain in the embryonic period, the cell group structure and overall function of the brain organoid model are more suitable for the human brain than rodents. Simulating organoids exposure to environmental toxins can provide models that are more accurate and predict the effects of these toxins on human tissues and organs than traditional two-dimensional cell culture.

2) High-throughput screening: organoids can grow in large numbers and be screened using high-throughput methods, enabling researchers to quickly and effectively test the effects of various environmental toxins on organoid behavior.

3) Personalized medicine: organoids can be derived from patient-specific stem cells, allowing personalized medicine methods to study the effects of environmental toxins on human tissues and organs.

4) Improve reproducibility: organoid-based detection can improve reproducibility by controlling environmental factors and batches, and traditional toxicological testing methods may be affected by factors such as differences between animal individuals.

5) Improve the understanding of disease mechanisms: organoids can be used to study the effects of environmental toxins on the occurrence and development of diseases. By exposing organoids to environmental toxins, researchers can gain insight into the underlying mechanisms of disease development and identify potential targets for therapeutic interventions, including through gene sequencing.

6) Developing new therapies: organoids can be used to test the efficacy and safety of new drugs and treatments for environment-related diseases, by using organoids to screen potential candidate drugs, researchers can identify the most promising treatments for further testing and development.

### 2. Cost Reduction

1) There is less demand for animal testing: organoid-based detection methods can be cheaper than traditional animal models used to study the effects of environmental toxins. This can also reduce the need for animal testing, which is a major ethical issue.

2) Reduced time and resources: simulating organoid contact materials can significantly reduce the time and resources required for toxicological testing. Traditional toxicological testing methods are time-consuming and expensive, and organoid-based detection can quickly and effectively screen environmental toxins.

### 3. Ethical Aspect

1) Safer for researchers: traditional methods of studying the effects of environmental toxins on human tissues and organs may be harmful to researchers. Organoids grow in vitro, reducing the risk of exposure to toxic substances.

2) Reduce or even replace animal experiments, and reduce the need for animal testing.

3) Brain organoids can simulate the process of embryonic development of the brain, detect the effect of environmental exposure on brain development after intervention, and study its mechanism, which cannot be carried out in real experiments.

In general, simulating the exposure of brain organoids to environmental exposures is a promising method to study the effects of environmental toxins on human health. Compared with traditional toxicological testing methods, this technology has many advantages and may improve our understanding of environmental-related diseases and help to develop new strategies for the prevention and treatment of these diseases.

### BRIEF DESCRIPTION OF DRAWINGS

FIG. 1 is an implementation scheme of an example of the present invention.

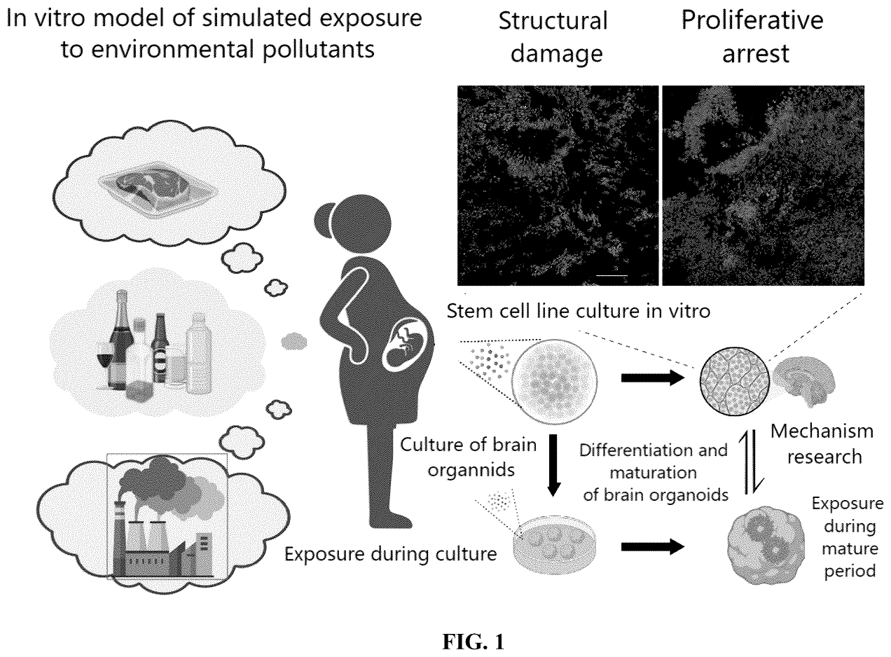

FIG. 2 shows a white light imaging of brain organoids under the microscope provided by an embodiment of the invention, it can be seen from the white light imaging that the organoids grow well and can grow stably; as well as the pictures after the intervention of environmental pollutants, the pictures can directly see the impact of environmental pollutants on the growth and development of brain organs;

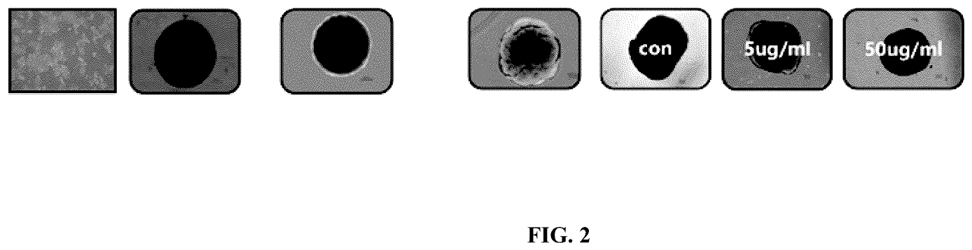

FIG. 3 is a frozen section of the cultured organoids and the immunofluorescence experiment. As shown in the figure, brain organoids include at least group cells (SOX2⁺ cells), neurons (MAP2⁺ cells), deep neurons (TBR1⁺ cells) and immature neurons (TUJ1⁺ cells), which have a relatively complete cerebral cortex structure, forming a ‘miniature landscape’ of brain development similar to that of embryonic period.

FIG. 4 shows an example of the invention, after the intervention of environmental pollutants, the immunofluorescence staining of brain organoids after frozen sections contains growth and apoptosis-related markers, these indicators characterize the cell growth and apoptosis levels during the growth and development of brain organoids.

FIG. 5 shows a gene expression at the transcriptome level.

FIG. 6 is a differential gene enrichment pathway.

### DETAILED DESCRIPTION OF THE EMBODIMENTS

In order to better explain the present invention, in order to facilitate understanding, the following combined with the attached figure, through the specific implementation of the invention for a detailed description.

In order to better understand the above technical scheme, the following will refer to the attached diagram to describe the example embodiment of the invention in more detail. Although the illustrated embodiments of the invention are shown in the figures, it should be understood that the invention can be realized in various forms and should not be limited by the embodiments described. On the contrary, these embodiments are provided in order to understand the invention more clearly and thoroughly, and to convey the scope of the invention to the technical personnel in the field.

The human embryonic stem cells involved in the embodiment of the invention are human embryonic stem cell line H9, the cell line H9 used in the invention is a mature and commercialized cell line that has been established. The invention does not involve the steps of obtaining embryonic stem cells from human embryos or blastocysts, and does not involve the application of human embryos for industrial or commercial purposes.

The brain organoid model constructed by the invention is applied to the study of brain development damage caused by environmental pollutants in the simulated embryonic period, the results obtained are only used as intermediate results for the prevention or treatment of diseases, the brain organoid model of the invention can only be used to evaluate the impact of environmental pollutants on the brain in the embryonic period, and is not used as a basis for the diagnosis and treatment of a disease.

### Example 1: Culture and Preparation Of Brain Organoid Model

When the specific experimental temperature is not indicated in the embodiment of the invention, the experimental temperature is room temperature (20-25° C.) . The sources of reagents used in the embodiment of the invention are as follows:

DMEM/F12 substratum is purchased from GIBCO, USA, Neurobasal is purchased from GIBCO, USA, Glutamax is purchased from Thermo Fisher Scientific, USA, NEAAs is purchased from Thermo Fisher Scientific, USA, Rho-associated protein kinase (ROCK) inhibitor Y27632 is purchased from Stem Cell Technologies, Canada, Vitamin A-free cell culture additive B-27 is purchased from Thermo Fisher Scientific, USA, Penicillin-streptomycin is purchased from SIGMA, USA, and Insulin is purchased from MedChemExpress, USA.

The substratum used in the implementation of the invention is self-prepared, and the components include: 0.48 volume of DMEM/F12 substratum, 0.48 volume of Neurobasal substratum, 0.01 volume of cell culture additive Glutamax, 0.01 volume of non-essential amino acids NEAAs, 0.01 volume of cell culture additive B27, 0.01 volume of penicillin-streptomycin, and 0.0025 volume of insulin.

The human embryonic stem cell line H9 is seeded on a Matrigel-coated cell culture plate (Corning, New York, USA) and incubated in 5 % CO₂ at 37° C., the cells are passaged once every four days and cultured in mTeSR plus serum-free substratum. In order to induce neuroectoderm derivation in 3D culture system, human embryonic stem cell line H9 is seeded at a density of 9×10³ cells/well in an ultra-low-parallel 96-well plate to form embryoids (EBs). A high concentration (50 μM) of ROCK inhibitor Y27632 is added to promote the formation of EB. The substratum is changed every other day until the 10th day of differentiation. After 3D culture to induce neural differentiation during this period, EB is allowed to differentiate into cortical organoids (HCO), which can mimic the cortical tissue of the embryonic brain. On the 10th day, EB is transferred from ultra-low attachment 96-well plates to ultra-low attachment 6-well plates using a 5 mL pipette, and the substratum is replaced with a neural differentiation medium without vitamin A. On the 18th day, neural differentiation substratum is used for neural maturation and long-term culture. The brain organoid model is constructed.

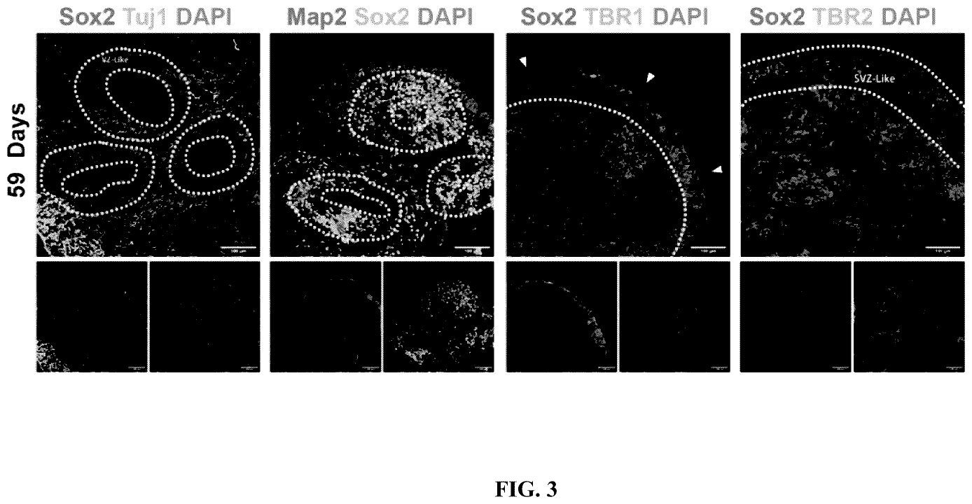

The above-mentioned cultured brain organoid model is frozen sectioned and immunofluorescence experiments are performed. As shown in FIG. 3, the brain organoid model includes at least group cells (SOX2⁺ cells), neurons (MAP2⁺ cells), deep neurons (TBR1⁺ cells) and immature neurons (TUJ1⁺ cells), that is, it has a relatively complete cerebral cortex structure. Compared with the animal model, it is closer to the natural brain tissue in histology, forming a good in vitro research disease model.

### Example 2: Effect of Environmental Pollutants On Brain Organoid Model

In order to verify that the brain organoid model constructed in example 1 may provide a reliable model for studying environmental pollutants, the invention realizes the simulated exposure environment by adding environmental pollutants (taking PM2.5 as an example) to the brain organoid model medium, studies the inhibitory effect of environmental pollutants on the brain organoid model, and detects the level of proliferation and apoptosis at the cellular level.

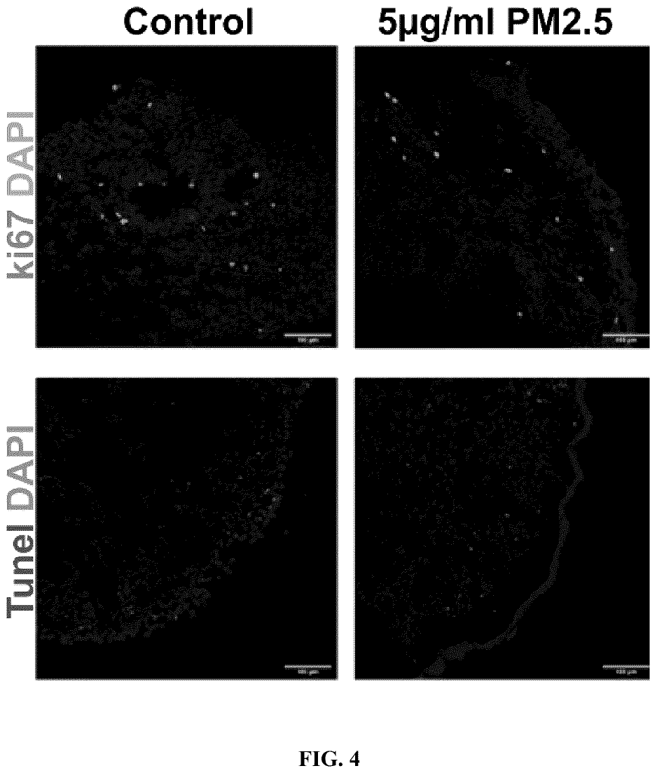

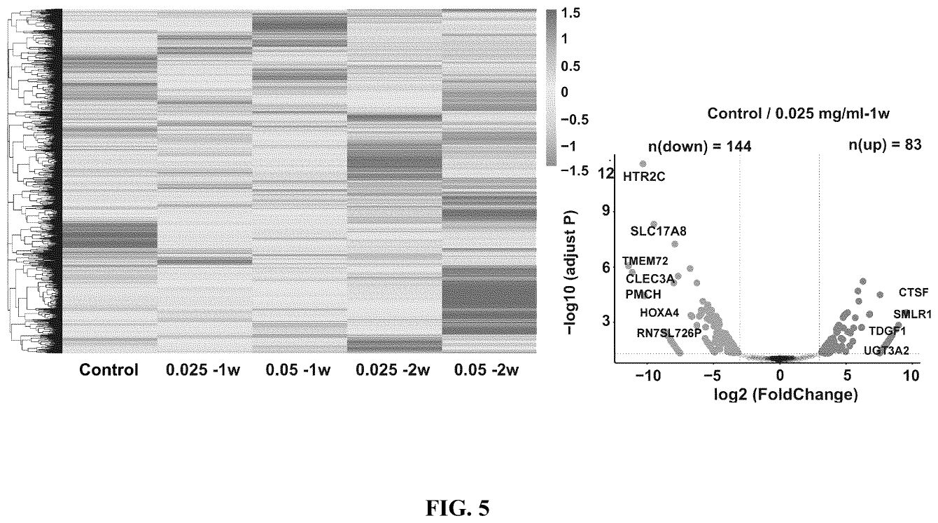

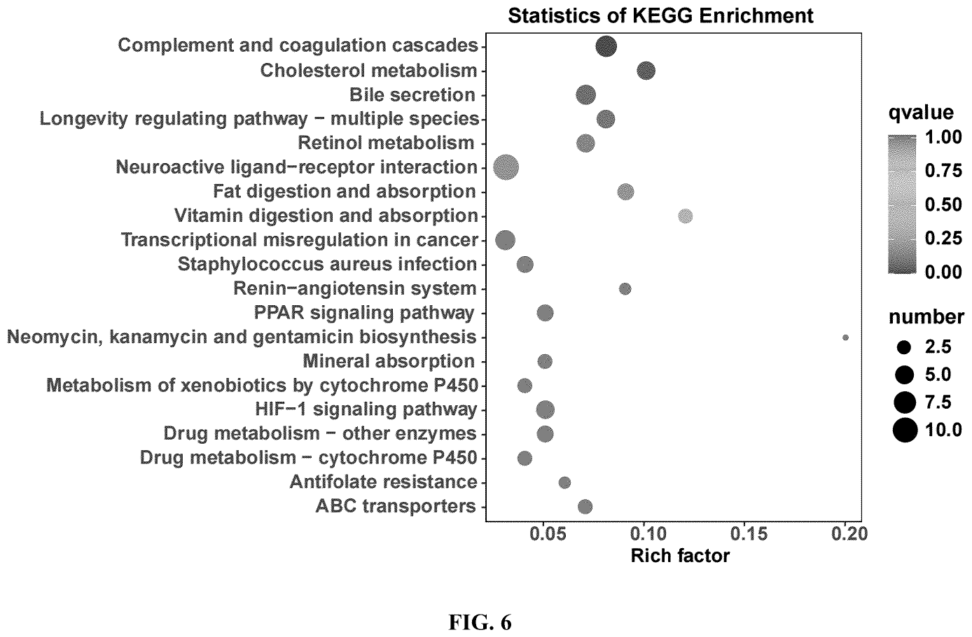

First, the present invention analyzes the expression of Ki67, a proliferation marker, using immunofluorescence. Untreated controls showed significant expression of the proliferation marker Ki67. However, after the intervention of environmental pollutants, inhibition of Ki67 expression was observed in brain organoid cells, as shown in FIG. 4. In addition, TUNEL staining analysis was performed to confirm the inhibitory effect of environmental pollutants on brain organoid models. The results showed that environmental pollutants significantly inhibited the growth of brain organoids at the cellular level. In order to study the mechanism of environmental pollutants affecting human cortical brain organoids, transcriptional analysis of brain organoids was performed before and after exposure to different concentrations of environmental pollutants (FIG. 5 and FIG. 6). It can be seen from FIG. 5 that a large number of genes were down-regulated after the intervention of environmental pollutants. It can be seen from FIG. 6 that some growth and development-related pathways are significantly down-regulated after the intervention of environmental pollutants, once again showing the effect of environmental pollutants nanoplastics on brain-like organs. Sequencing results showed that a large number of genes were regulated at least twice in brain organoids. This difference indicates that the duration of treatment has a significant effect on the gene expression profile in the organoids. After the intervention of environmental pollutants, the genes regulating cell proliferation were significantly down-regulated in brain organoids.

### Example 3 High-Throughput Screening of Drugs That Reverse the Pathological Regulation of Environmental Pollutants

Based on the above, transcriptome sequencing technology is used to identify differentially expressed genes and the enrichment of these genes in specific pathways. Further examine the key genes of these pathways, and further high-throughput screening of agonists and inhibitors of corresponding potential targets to develop drugs that reverse the pathological regulation of environmental pollutants.

The brain organoid model used for drug screening is mixed in the corresponding medium at a specific exposure period (in this case, the exposure of the simulated substratum period, that is, the formula of the mature substratum used above), and inoculated into multiple 96-well culture dishes using a multi-channel pipette or continuous dispenser (depending on the number of drugs involved in screening), so that the volume of the brain organoid model and the substratum mixture per well is equal. Test drugs (agonists or inhibitors of target genes, or other target drugs with potential therapeutic effects) are added to the 96-well plate. Taking the WNT pathway as an example, the most significant down-regulated gene β-Catenin agonist SKL2001 and inhibitor LF3 can be used as target drugs. Each hole is a drug, and each drug is set up parallel repeat holes (the number of repeat holes range: 3-5), and blank control, environmental pollutant treatment without drug control group (control group without any substance or drug solvent with the same volume as the drug test hole), environmental pollutant+drug treatment group. After two weeks of mixed culture of brain organoid model and drug, light microscope observation and immunofluorescence evaluation were carried out to screen out the best drug that can block the lesions in brain development caused by environmental pollutants. This method can achieve high-throughput screening of target drugs in order to find therapeutic drugs more quickly to reduce the impact of environmental pollutants on human health.

The above implementation examples only express several implementation methods of the invention, and their descriptions are more specific and detailed, but they cannot be understood as restrictions on the scope of invention patents. It should be pointed out that for the ordinary technical personnel in this field, some deformations and improvements can be made without breaking away from the idea of the invention, which are all within the scope of protection of the invention. Therefore, the scope of protection of the invention patent should be subject to the attached claims, and the instructions and drawings can be used to explain the content of the claims.

What is claimed is:

1. An application of a brain organoid model for simulating the damage of environmental pollutants to brain development during embryonic period.

2. The application according to claim 1, wherein the brain organoid model has a cerebral cortex structure, and the brain organoid model includes at least: group cells: SOX2⁺ cells, neurons: MAP2⁺ cells, deep neurons: TBR1⁺ cells and immature neurons: TUJ1⁺ cells.

3. The application according to claim 1, wherein an embryonic period is the human embryonic period.

4. The application according to claim 1, wherein the environmental pollutants include the hazards caused by production, construction or activities.

5. The application according to claim 4, wherein the environmental pollutants comprise:
endocrine disrupting chemicals, pharmaceuticals and personal care products, perfluorinated compounds, brominated flame retardants, disinfection by-products in drinking water.

6. The application according to any of claims 1-4, wherein a construction method of the brain organoid model comprises: culturing human embryonic stem cells in serum-free human embryonic stem cell culture medium to form embryoid bodies; then adding a ROCK inhibitor, after a certain time of differentiation, the embryoids are differentiated into cortical organoids, then replaced with vitamin A-free neural differentiation medium for culture, and obtaining a product.

7. The application according to claim 6, wherein the human embryonic stem cells are human embryonic stem cell line H9.

8. The application according to claim 6, wherein the ROCK inhibitor is Y27632.

9. The application according to claim 6, wherein a concentration of the ROCK inhibitor is 50-60μM.

10. The application according to claim 6, wherein the differentiation time is 10-12 days.
